(19) United States
(12) Patent Application Publication
(10) Pub. No.: US 2012/0195971 A1
(43) Pub. Date: Aug. 2, 2012

(54) Method for Preparing Mechanically Macerated Demineralized Bone Materials and Compositions Comprising the same

(75) Inventors: Nicholas Missos, Winona Lake, IN (US); Mark Vandewalle, Pierceton, IN (US)

(73) Assignee: Biomet Manufacturing Corp., Warsaw, IN (US)

(21) Appl. No.: 13/016,524

(22) Filed: Jan. 28, 2011

(51) Int. Cl. A61K 35/32; A61P 19/08

(52) U.S. Cl. 424/549

(57) ABSTRACT

The present invention relates generally to methods for preparing mechanically macerated demineralized bone materials that are useful in, or as, implants having a variety of orthopedic applications. More particularly, the demineralized bone materials prepared according to the present invention comprise web-like sections that are compression- and extrusion-resistant and thus, suitable for use in, or as, bone implants or bone grafting materials. Additionally, the web-like morphology increases the surface area of the graft and thereby facilitates the rehydration of the graft, should it be freeze dried. The increased surface area also allows for increased contact with autologous growth factors. The present invention further relates to the demineralized bone materials prepared by the disclosed methods and compositions comprising the same. Methods for preparing a bone void for orthopedic applications, such as bone implants, using the demineralized bone materials or compositions thus prepared are also provided.

### FIELD OF THE INVENTION

The present invention relates generally to methods for preparing mechanically macerated demineralized bone materials that are useful in, or as, implants having a variety of orthopedic applications. More particularly, the demineralized bone materials prepared according to the present invention comprise web-like sections that are compression- and extrusion-resistant and thus, suitable for use in, or as, bone implants or bone grafting materials. Additionally, the web-like morphology increases the surface area of the graft and thereby facilitates the rehydration of the graft, should it be freeze dried. The increased surface area also allows for increased contact with autologous growth factors. The present invention further relates to the demineralized bone materials prepared by the disclosed methods and compositions comprising the same. Methods for preparing a bone void for orthopedic applications, such as bone implants, using the demineralized bone materials or compositions thus prepared are also provided.

### BACKGROUND OF THE INVENTION

The skeletal structure of mammalian anatomy is strong enough to maintain its rigidity and structure through a given amount of force or use. However, the skeletal structure may be weakened or damaged for a variety of reasons. A bone repairing composition or filler can be used to correct bone defects caused by, for example, trauma, pathological disease, surgical intervention, or other situations where defects need to be managed in osseous surgery. A variety of bone repair materials and compositions have been described to aid in the repair or reconstruction of such bone defects.

Among the materials that have been suggested for bone repair or reconstruction is bone graft, which is known to provide support, promote healing, fill bony cavities, separate bony elements such as vertebral bodies, promote fusion and stabilize the sites of fractures. There are at least three types of bone graft materials: xenogenic grafts (xenografts), allogenic grafts (allografts), and autografts. The use of xenografts, allografts, and autografts is well known in both human and veterinary medicine. See e.g., Stevenson et al., Clinical Orthopedics and Related Research, 323: 66-74 (1996).

One form of bone graft is demineralized bone, which is typically formed through chemical treatment of bone so as to remove most or all of its mineral content. The remaining demineralized bone material consists of the native collagen structure of the bone, along with naturally-occurring growth factors, e.g., bone morphogenetic proteins. The demineralized bone material is both osteoconductive and osteoinductive. Osteoconduction is the promotion of differentiated bone-forming cells growth or infiltration into the demineralized bone material from the subject in whom the demineralized bone material is implanted, which means that the demineralized bone material supports the formation of new bone by acting as a matrix or scaffolding for extension or apposition of new bone from existing bone (i.e. the patient's own bone). Osteoinduction, on the other hand, is the promotion of new bone-forming cell production, from non-differentiated cells, in and around the implanted demineralized bone material, which means that in addition to acting as a simple scaffolding, the demineralized bone material may actually stimulate the patient's own mesenchymal cells to transform into osteoblasts (bone-forming cells) hastening the replacement of the graft material with the patient's own bone.

Special physical properties of the demineralized bone material may include, for example, (1) volume change with hydration, elasticity to compression, tension, torsion, bending, and compressibility; (2) different physical characteristics depending on whether the demineralized bone materials are hydrated or non-hydrated; (3) capable of mixing with blood, bone marrow aspirate, blood products, and the like; (4) expanding with hydration to fill a void without losing its tensile properties; (5) will not lose its tensile properties when infiltrated by body fluids; and (6) capable of absorbing blood and fluid and expand with hydration. One of the continuous challenges in preparing demineralized bone materials is to provide demineralized bone materials that will absorb more fluid upon hydration.

In manufacturing demineralized bone material, generally, the harvested bone is subdivided into sections of whole, i.e., mineralized, bone by mechanical operations such as shredding, milling, shaving, and/or machining, and undergoes physical processing such as grinding or shaping, and is then demineralized to form demineralized bone material by, for example, treatment with acid. For example, Campbell T. D. (U.S. Pat. No. 5,053,049) discloses a method for producing flexible prostheses of predetermined shapes derived from bone by machining a bone segment into the desired shape, demineralizing the bone segment to impart the desired degree of flexibility, followed by tanning to render the material non-antigenic, biocompatible, and stabilized. Likewise, Gendler et al. (U.S. Pat. No. 5,306,304 and progenies thereof) discloses a method for producing a flexible organic bone sheet of demineralized natural bone by machining the bone to produce the sheet followed by demineralizing the sheet.

The demineralized bone material may be subjected to further mechanical shaping using forces, such as pressing, extruding, and/or rolling. For example, Boyce et al. (U.S. Pat. No. 6,863,694) discloses a method of preparing an osteogenic osteoimplant in the form of a flexible sheet by mechanically shaping demineralized bone material to form such a flexible sheet, which may be accomplished through application of compressive and, optionally, simultaneous lateral forces. Devices for shaping or compressing tissues such as bone or graft tissue are disclosed in, for example, Bonutti P. M. (U.S. Pat. No. 5,329,846).

The demineralized bone material also may be transformed between different configurations because it is compressible, expandable and may be rehydrated. For example, Boyer et al. (U.S. Pat. No. 7,608,113) discloses a method of providing a bone implant by demineralizing a block of bone having a first geometry followed by wetting and compressing the block to form a second geometry which is smaller than the first geometry, after hardening the block with the second geometry, re-expanding the block to a third geometry that is larger than the second geometry.

In preparing bone implants or graft materials, the demineralized bone material is often used to provide flexibility. For example, Sunwoo et al. (U.S. Pat. No. 6,998,135) discloses a sterile flexible bone sheet comprising a sheet of demineralized bone with a cortical layer and a cancellous layer. Including a cortical bone layer with a demineralized portion to give flexibility to a load-bearing bone strip implant is also disclosed in Bindsell et al. (U.S. Pat. No. 7,537,617).

Because the bone may be harvested and processed in advance of its use, it is frequently dried (e.g., freeze-dried by lyophilization) and packaged under sterile conditions, for storage and/or shipping to the clinical site. The resulting dried, e.g., freeze-dried, demineralized bone material can then be used alone or mixed with additives or carriers to form gels, putties, and sheets as bone filler. See e.g., Urist, M. R., Science, 150: 893-899 (1965) and Maddox et al., Tissue Eng., 6: 441-448 (2000). Compounds such as polyhydroxyl compounds, polysaccharides, glycosaminoglycan proteins, nucleic acids, polymers, polaxomers, resins, clays, calcium salts, and/or derivatives thereof may be used as additives or carriers to mix with freeze-dried demineralized bone material to form a bone filler composition. See e.g., Knaack et al., U.S. Pat. No. 5,507,813. However, because bone defects are usually jagged or irregularly shaped, it is important to have the bone filler of an appropriate composition to facilitate placement of the filler into the surgical site.

For example, phospholipids such as lecithin are used as carriers to mix with freeze-dried demineralized bone material to form a putty-like grafting material. Han et al., “Lecithin Enhances the Osteoinductivity of DBM,” Poster No. 0509, 48th Annual Meeting of the Orthopaedic Research Society, Dallas, 2002. However, although lecithin blended with demineralized bone material is reported to provide better handling properties and have the ability to enhance the osteoinductivity of demineralized bone material (see Han et al., 2002), it has a tendency to be displaced from the placement site upon compression.

Accordingly, it would be advantageous to provide demineralized bone materials that can be efficiently rehydrated and used as bone filler and are compression- and extrusion-resistant. It would be further advantageous to provide a bone repairing material and/or composition that is easily placed into an injury site, adheres to the injury site, and is not easily displaced upon compression.

### SUMMARY OF THE INVENTION

The present invention provides a method for preparing mechanically macerated demineralized bone materials that are useful in, or as, implants having a variety of orthopedic applications.

In one aspect, the invention provides a method for preparing a demineralized bone material useful for orthopedic applications, comprising:

a) demineralizing a strip of bone;

b) compressing the strip of bone in a press to create a flexible web-like section; and

c) preserving the strip of bone.

The strip of bone may be obtained from various sources, such as an animal source or a human source. The strip of bone may be obtained from cortical, cancellous and/or corticocancellous bone. The strip of bone may be obtained lengthwise along a longitudinal axis of the bone.

The strip of bone is compressed in a press to create a flexible web-like section. In one preferred embodiment, the strip of bone is compressed with a hydraulic press comprising two metal press plates or a plastic press plate opposed from a metal press plate to create a flexible web-like section. In another preferred embodiment, at least one of the two metal press plates or one of the plastic press plate and the metal press plate includes a textured surface to form a textured area on the demineralized bone material.

The compressed strip of bone is then preserved. In one embodiment, the strip of bone is preserved by freeze-drying. In another embodiment, the strip of bone is preserved by freezing. In yet another embodiment, the strip of bone is preserved by liquid storage in, for example, acid, ethanol, or saline.

In a further embodiment, the strip of bone may be cut into a desired shape or form prior to the demineralizing step or the compressing step of any of the aforementioned methods. In yet another embodiment, the aforementioned methods additionally may comprise shaping and forming the demineralized bone material thus obtained to fit a particular bone void.

In another aspect, the invention provides a demineralized bone material with flexible web-like section that is resistant to compression and extrusion obtained according to any of the aforementioned methods.

In yet another aspect, the invention provides a composition useful for orthopedic applications comprising the demineralized bone material obtained according to any of the aforementioned methods and at least one carrier. In one embodiment, the at least one carrier is a phospholipid. In another embodiment, the at least one carrier is lecithin.

In a further aspect, the invention provides bone implants or bone grafting materials comprising the demineralized bone material obtained according to any of the aforementioned methods or a composition comprising the demineralized bone material thus obtained.

In a yet further aspect, the invention provides an implant for use in bone reconstruction comprising the demineralized bone material obtained according to any of the aforementioned methods or a composition comprising the demineralized bone material thus obtained.

In yet another further aspect, the invention provides a method for preparing a bone void for orthopedic applications, comprising:

a) obtaining a demineralized bone material according to any of the aforementioned methods or a composition comprising the demineralized bone material thus obtained;

b) shaping and/or forming the demineralized bone material into a bone void; and

c) optionally rehydrating the demineralized bone material.

In one embodiment, the demineralized bone material is rehydrated with an aqueous solution or fluid containing water. In another embodiment, the demineralized bone material is rehydrated with corpus fluids, blood, or blood components.

The methods and compositions of the present disclosure provide benefits over methods and compositions among those known in the art. Such benefits can include, but not limited to, affording a bone graft material that is not easily displaced from the site to which it is placed or implanted, even upon compression or in the presence of body fluids and after the passage of time, has enhanced strength, and is resistant to dissolution by blood or other fluids and easy hydration. Further areas of applicability will become apparent from the detailed description provided hereinafter. It should be understood that the Detailed Description and specific examples, while indicating preferred embodiments of the present disclosure, are intended for purposes of illustration only and not intended to limit the scope of the present disclosure.

### BRIEF DESCRIPTION OF THE DRAWINGS

The present disclosure will become more fully understood from the detailed description and the accompanying drawings, wherein:

FIG. 1 depicts a flow chart illustrating a method according to the invention.

FIG. 2 depicts a flow chart illustrating a method for adjusting pH of demineralized bone material.

FIG. 3 depicts a strip of bone prior to demineralization (FIG. 3A) and a demineralized bone material obtained according to a method of the invention (FIG. 3B).

FIG. 4 depicts various surfaces on the press plate: polished plate (FIG. 4A), textured plate (FIG. 4B), grooved plate (FIG. 4C), and waffled plate (FIG. 4D).

FIG. 5 depicts one of the various possible applications of the demineralized bone materials obtained according to any of the aforementioned methods, in which the demineralized bone materials are placed together (FIG. 5A), rolled into a cylinder shape (FIG. 5B), and placed into a syringe-like hydration tube for rehydration (FIG. 5C).

### DETAILED DESCRIPTION OF THE PREFERRED EMBODIMENTS

The following description of the embodiment(s) is merely exemplary in nature and is in no way intended to limit the invention, its applications, or uses.

Throughout this application, various publications are referenced. The citation of references does not constitute an admission that those references are prior art or have any relevance to the patentability of the disclosure provided herein. Any discussion of the content of references cited in the present disclosure is intended merely to provide a general summary of assertions made by the authors of the references, and does not constitute an admission as to the accuracy of the content of such references. The disclosures of all of these publications and those references cited within those publications are hereby incorporated by reference in their entireties into this application in order to more fully describe the state of the art to which this invention pertains. The terminology used herein is for the purpose of describing specific embodiments only and is not intended to be limiting. As used herein, “a” or “an” can mean one or more, depending upon the context in which it is used. Thus, for example, reference to “a strip of bone” can mean that at least one strip of bone can be used. When the term “about” is used in conjunction with a numerical range, it modifies that range by extending the boundaries above and below the numerical values set forth. In general, the term “about” is used herein to modify a numerical value above and below the stated value by a variance of 20%, preferably 10% up or down (higher or lower). The word “comprise,” “comprising,” “include,” “including,” and “includes” as used herein and in the following claims is intended to specify the presence of one or more stated features, integers, components, or steps, but they do not preclude the presence or addition of one or more other features, integers, components, steps, or groups thereof.

In one aspect, the invention provides bone derived materials and compositions for the treatment of bone defects in humans or animals. Specific materials to be used must, accordingly, be biocompatible. As used herein, such a “biocompatible” component is one that is suitable for use with humans and/or animals without undue adverse side effects (such as toxicity, irritation, and allergic response) commensurate with a reasonable benefit/risk ratio.

In various embodiments, bone derived materials and compositions according to the present disclosure comprise demineralized bone materials that are prepared and/or made by a method comprising: (a) demineralizing a strip of bone; (b) compressing the strip of bone in a press to create a flexible web-like section; (c) preserving the strip of bone. Optionally, the demineralized bone materials thus prepared is packaged, sterilized, and/or irradiated for future uses.

As used herein, “demineralized bone material(s)” refers to any bone material(s) that is derived from human and/or animal, from which naturally-occurring minerals have been removed in whole or in part. Such bone material includes bone powders and other bone constructs such as cubes, rods, dowels, pins, disks and other formed devices. In one embodiment, the bone material has been entirely demineralized. In another embodiment, only a substantial portion of naturally-occurring minerals of the bone material has been removed. Preferably, the mineral content of the demineralized bone is less than about 20% by weight, optionally less than about 10% by weight, optionally from about 0% to about 5% by weight, optionally from about 0% to about 2% by weight, optionally less than about 0.5% by weight.

As used herein, “strip(s) of bone” refers to bone segment(s) or strip(s) that is(are) obtained by milling, cutting, shaving, grinding, crushing, or other equivalent means, a bone into segment(s) or strip(s) of various lengths, widths, and thicknesses. The strip(s) of bone can be in any shape, such as rectangular or in the shape of a toothpick, but the final shape of the strip(s) of bone depends largely on the shape of the original bone material obtained.

In one embodiment, bone is obtained from animal sources such as cows and pigs for, for example, xenogenic graft in a human subject. In a preferred embodiment, bone is obtained from human cadavers (i.e., for allogenic graft in a human subject), following appropriate ethical and legal requirements. Such human bone material is available from a variety of tissue banks. In another preferred embodiment, bone is autologous bone or donated from a single member of the same species as the patient to reduce or prevent an immunogenic response. For example, all of the bone used to prepare the bone material according to the present disclosure for a human patient may be sourced from a single human cadaveric donor. However, bone from multiple donors may also be used.

Bone used in embodiments according to the present disclosure may include the entire bone or bone fragments from cortical, cancellous and/or corticocancellous bone. See e.g., Dowd, et al., U.S. Pat. No. 5,507,813. Preferably, the bone is cortical bone. Cortical bone may be obtained from long bones, such as the diaphyseal shaft of the femur and tibia.

After the bone is obtained from the donor, any adherent tissues can be removed from the bone by standard bone cleaning protocol. The bone is further processed, e.g., cleaned, disinfected, and/or defatted, using methods well known in the art. Depending on the desired end-use of the bone composition, the bone may be subjected to mechanical processing. Such processing may include cutting and shaping, in embodiments forming a construct such as a bone pin or disk for implanting. In various embodiments, the bone is milled into strips of various lengths, widths, and thicknesses. As used herein, the term “milled” and conjugations thereof, refers to shaping a tissue or a bone to the desired size by crushing, chopping, cutting, shaving, grinding, or pulverizing. In embodiments where several sizes of bone are to be used, it is understood that the milling process may be repeated and the respective bone portions may be reserved and assigned accordingly. Commercially available milling and sieving devices may be used, or bone may be purchased in the form of an allograft matrix in the desired particle size or sizes.

Milled bone may be defatted by soaking or washing the bone in ethanol because the high polarity of ethanol solubizes the less polar lipids. A preferred ethanol solution is at least 60% ethanol, volume to volume, in deionized/distilled water. A more preferred ethanol solution is 100% ethanol. The ethanol bath also disinfects the bone by killing vegetative microorganisms and viruses. A further antiseptic step may include treatment of the milled bone with a hydrogen peroxide solution.

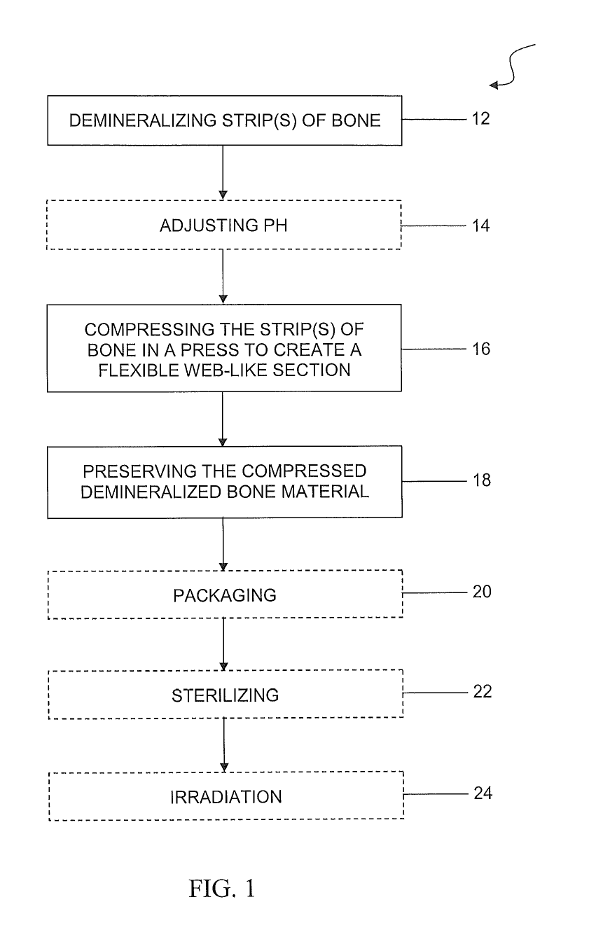

Referring next to FIG. 1, an embodiment of the method is illustrated by a flowchart 10. In a first step 12, strip(s) of bone is(are) demineralized.

To prepare the bone material, milled bone is demineralized using any of a variety of conventional procedures, including those known in the art using acids, chelating agents and electrolysis. Acids used include inorganic acids, such as hydrochloric acid, or organic acids, such as peracetic acid. Chelating agents include disodium ethylenediaminetetraacetic acid (Na_{z} EDTA). Preferred chemical treatments include those using hydrochloric acid, ethylene diamine tetraacetic acid (EDTA), peracetic acid, or citric acid. Demineralization techniques among those useful herein are described in Lewandrowski et al., J. Biomed. Mater. Res., 31: 365-372 (1996); Lewandrowski, et al., Cal. Tiss. Int., 61: 294-297 (1997); Lewandrowski, et al., J. Orthop. Res., 15: 748-756 (1997); and Reddi et al., Proc. Nat. Acad. Sci., 69: 1601-1605 (1972).

The strength of the acid solution, the shape and size of the bone and the duration of the demineralization procedure will determine the extent of demineralization. Generally, larger bone portions as compared to small particles will require more lengthy and vigorous demineralization. Guidance for specific parameters for the demineralization of different size bone can be found in Scarborough et al., U.S. Pat. No. 5,846,484, Harakas, Clin. Orthop. Relat. Res., 239-251 (1983), and Lewandrowski et al., J. Biomed. Mater. Res., 31: 365-372 (1996). As used herein, the term “demineralized” and variants thereof, means a loss or decrease of the mineral constituents or mineral salts of the individual tissues or bone relative to their natural state. Preferably, the demineralized bone has a calcium concentration not exceeding 8% according to the American Association of Tissue Banks (AATB) standards. More preferably, the demineralized bone has a calcium concentration of about 1%, less than 1%, or between 0.5% and 1%.

The time required to demineralize the bone may vary depending on the concentration of acid or chelating agent used, the displacement or flow of the solution, and the desired final concentration of calcium in the bone. For example, in an embodiment using hydrochloric acid, at an acid concentration of 0.1 to 2.0 M, the bones may be soaked in the acid bath for up to 24 hours. The calcium or mineral concentration in the milled bone may be monitored by measuring the pH of the acid solution using a calcium specific electrode or a standard pH meter. In a preferred embodiment, the acid wash or soak ceases when the calcium concentration of the bone is less than 1%.

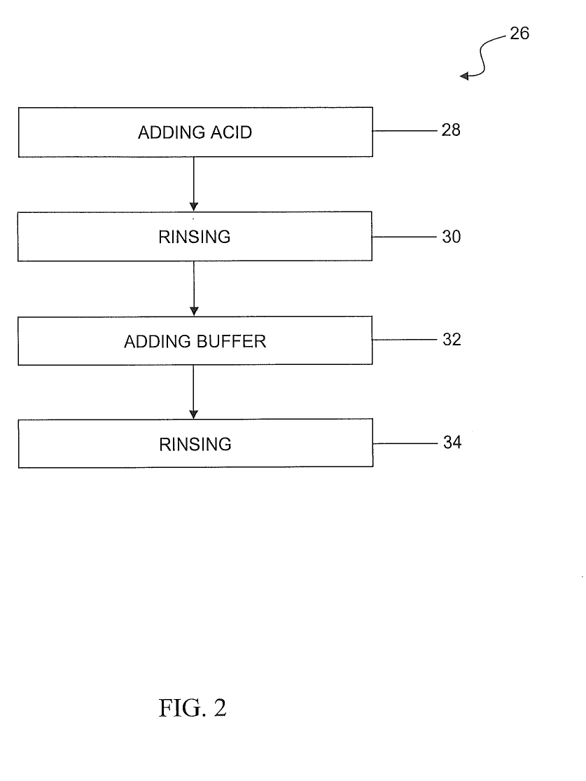

Referring again to FIG. 1, after demineralization (step 12), optionally the pH of the bone is adjusted (pH adjustment step 14) by removing the acid with a wash with deionized/distilled water until the pH of the bone approximates that of the water. The process of such a pH adjustment step is further illustrated by a flowchart 26 in FIG. 2. In a first step 28, acid is added to demineralize the bone material. The acid is rinsed off from the bone material with deionized/distilled water (rinsing step 30). The bone material is then put into a buffer to lower the pH (adding buffer step 32). The buffer is then rinsed off from the bone material with deionized/distilled water (rinsing step 34). It is not outside of the scope of embodiments of the present disclosure to expedite the neutralization of the bone using an ionic strength adjuster, such as a biocompatible buffer solution.

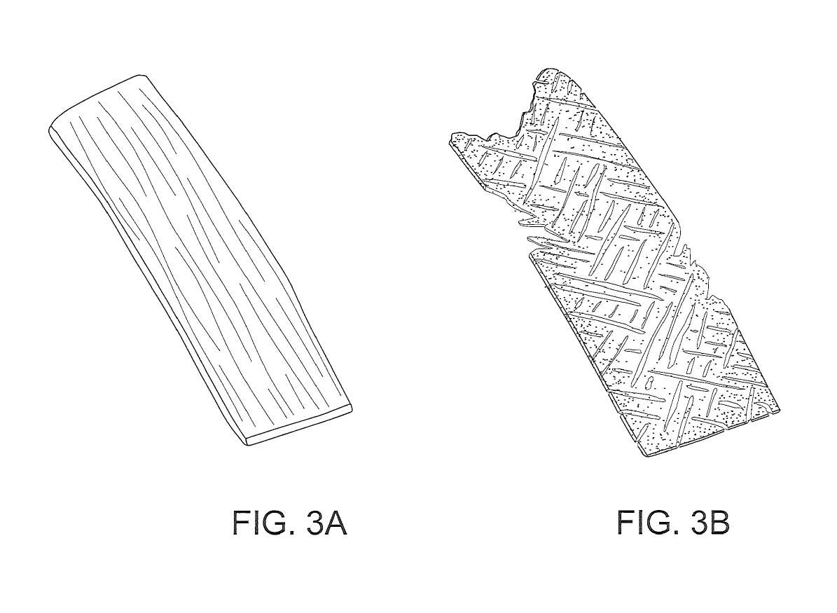

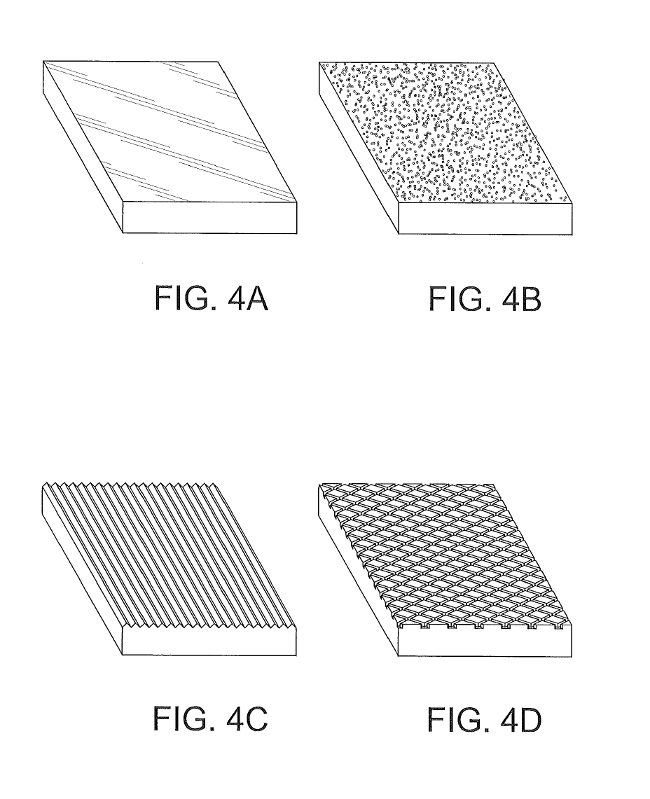

Following demineralization and optionally pH adjustment, the bone strip(s) is compressed (compressing step 16) to create a mechanically macerated flexible web-like section, which is essentially flexible collagen fiber. Any type of mechanical press, such as an arbor press or a hydraulic press, may be used. A press with plates made of metal, plastic, or any combination thereof, may be used. In preferred embodiments, the bone material is placed between the two plates of a hydraulic press and compressed. For example, the hydraulic press may comprise two metal press plates and the bone material is placed between the two metal plates and compressed. In another example, the hydraulic press may comprise one plastic plate and one metal plate, and the bone material is placed between the plastic plate and the metal plate and compressed. The plates may be smooth or textured to enhance pressing and/or crushing. FIG. 4 shows an example of polished plate (FIG. A), textured plate (FIG. B), grooved plate (FIG. 4C), and waffled plate (FIG. 4D). The bone material is compressed between 0 to 7,500 pounds per square inch (psi), preferably 1,000 to 6,000 psi, more preferably, 2,000 to 5,000 psi, even more preferably 3,000 to 4,000 psi to create a mechanically macerated flexible web-like section. In some embodiments, the starting section, before being compressed, may be under 80×10⁻³ inch of thickness (equivalent of about 2 mm). Preferably, the starting section is about 10-80×10⁻³ inch of thickness (equivalent of about 0.25-2 mm). More preferably, the starting section is about 40-80×10⁻³ inch of thickness (equivalent of about 1-2 mm). An example of such a web-like section so created is shown in FIG. 3B as compared to the strip of bone prior to the demineralization treatment (FIG. 3A).

The demineralized bone material is then preserved (preserving step 18) according to the methods known in the art for storage and/or further rehydration. Suitable preserving process may include, but not limited to, drying, freezing, and/or liquid storage. In one embodiment, the demineralized bone material is preserved by drying. Suitable drying techniques include, but not limited to, for example, freeze-drying, vacuum drying, air drying, temperature flux drying, molecular sieve drying, and other appropriate techniques. In preferred embodiments, the demineralized bone material is preserved by freeze-drying. As used herein, the term “freeze-drying” or “lyophilization,” and variants thereof, means the process of isolating a solid substance from solution by freezing the solution and evaporating the ice under a vacuum. The dried bone material has a final moisture level of about less than 6% as recommended by AATB. In another embodiment, the demineralized bone material is preserved by freezing. In yet another embodiment, the demineralized bone material is preserved by storing the demineralized bone material in liquid (i.e. liquid storage). Suitable storage liquids include, but not limited to, acid, ethanol, and saline.

The process of the present invention optionally comprises other steps. For example, the strip of bone used in the aforementioned methods may be further cut into a desired shape or form prior to the demineralizing step 12 or the compressing step 16. Additionally, the demineralized bone material obtained according to any of the aforementioned methods may proceed to further processes including, but not limited to, packaging (packaging step 20), sterilization (sterilizing step 22), and irradiation (irradiation step 24) according to the methods known in the art. Such additional process steps may be performed without a particular order. For example, the bone material may be packaged prior to sterilization and/or irradiation. Likewise, the bone material may also be sterilized prior to packaging and/or irradiation. Sterilization includes irradiation or chemical sterilization techniques (such as using ethylene oxide). Packaging includes methods suitable for convenient storage, handling or transport of the bone material after preparation and before use. Irradiation may be performed at any radiation dosages required for obtaining a sterility assurance level for a particular device and can be determined from the “Association for the Advancement of Medical Instrumentation Guidelines” published in 1992. A non-limiting example of irradiation of bone material is provided in US 2010/0172954.

The compressed, demineralized bone material may be further shaped to form a structure that fits a particular bone void. The shaping may be performed prior to the preserving step 18 by compressing the bone material into, for example, a cylinder shape with the aid of demineralized gel carrier, such as bone powder from the same donor mixed with saline. The shaping may also be performed during the preserving step 18 with the aid of a freeze-dry mesh to shape the bone material in roll. The shaping is not limited to roll shape or cylinder shape, it may also be half-cylinder shape or rectangular shape. However, the shaping is generally limited by the shape of the starting material and also depends on the final use of the bone material. It may also be re-shaped on site by surgeon or medical professional.

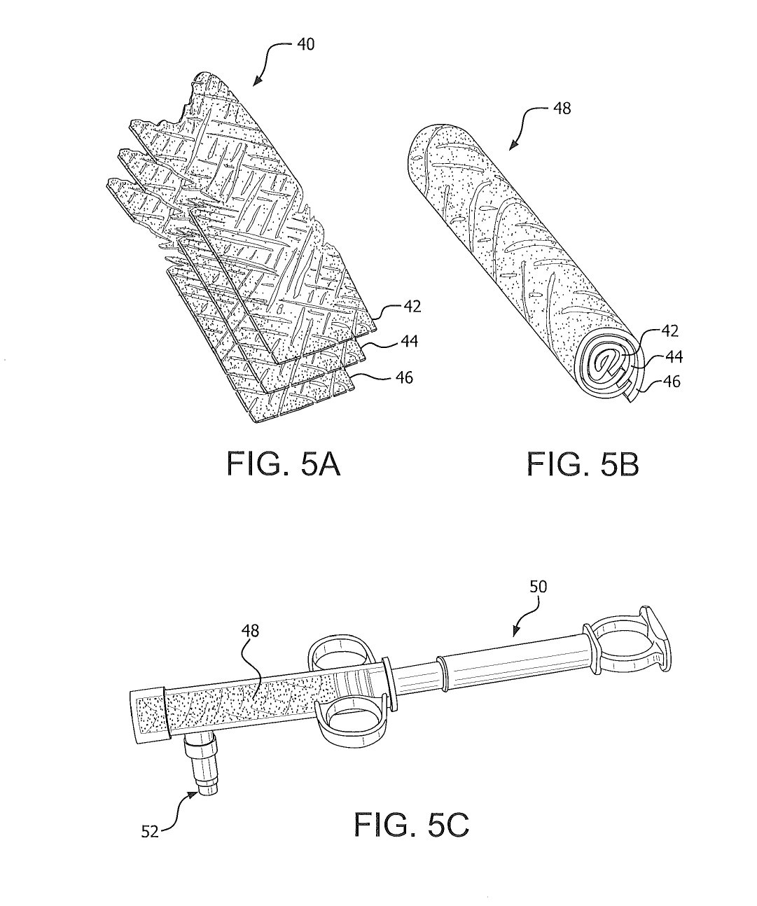

FIG. 5 exemplifies such an application. Several compressed, demineralized bone materials (42, 44, 46) are placed together (40; FIG. 5A) and rolled into a roll shape or cylinder shape (48; FIG. 5B). The roll-shaped or cylinder-shaped demineralized bone materials are then placed into a syringe-like hydration tube (50) for rehydration (FIG. 5C) as discussed below.

The freeze-dried demineralized bone material may be optionally allowed to rehydrate by adding an aqueous solution or fluid preferably containing water. The rehydration may be performed by adding ambient fluids such as blood to the dried bone. Hydration blood includes, but is not limited to, whole blood and blood components such as, red blood cells and components, white blood cells and components, plasma, plasma fractions, plasma serum, platelet concentrate, blood proteins, thrombin, and coagulation factors. The rehydration may also be performed by adding extra corpus fluids or aqueous-based liquids, such as, but not limited to, saline, water, or a balanced salt solution (e.g., 140 mM NaCl, 5.4 mM KCl, pH 7.6), or the like. In some embodiments, the bone material is rehydrated by water or saline solution in a syringe to allow rehydration. Biologically active materials (e.g., therapeutic and/or prophylactic), such as, but not limited to, antibiotics, platelet concentrates, bone growth factors, bone proteins, may also be included in the fluid or liquid used for rehydration. Additionally, additives or carriers may also be mixed with the demineralized bone material to form a composition suitable for bone graft, implanting, or any other suitable orthopedic applications. Carriers suitable for such uses are disclosed in, for example, Kumar et al., U.S. Pat. No. 7,670,384. Various hydration apparatus, such as that disclosed in Kumar et al. (U.S. Pat. No. 7,670,384), can also be used to facilitate hydration of the bone material. As used herein, the term “rehydrated,” and variants thereof, means to include at least either hydrated and/or rehydrated.

Returning to FIG. 5, which illustrates a possible way by which the demineralized bone materials may be rehydrated. As mentioned above, the demineralized bone materials are placed together (40; FIG. 5A), rolled into a roll shape or cylinder shape (48; FIG. 5B), and placed into a syringe-like hydration tube for rehydration (50; FIG. 5C). The aqueous solution or fluid suitable for rehydrating the demineralized bone materials is then added to the compartment containing the roll-shaped or cylinder-shaped demineralized bone materials 48 through the valve 52 during the rehydration process.

It should be understood that the final shape of the demineralized bone materials may vary depending on the application. It should also be understood that the rehydration may be performed in any type of enclosed containers into which the aqueous solution or fluid suitable for rehydration may be added. Examples of such containers include, but not limited to, a syringe-like hydration tube as exemplified in FIG. 5, a tray, or a pouch.

Regardless whether it is used alone as void filler or mixed with additives or carriers to form a composition, the demineralized bone material according to the present disclosure can be used to repair bone defects. As used herein, “bone defects” or “injury sites”, and variants thereof, refer to bone imperfections caused by birth defect, trauma, disease, decay, or surgical intervention, and the desired repair can be for cosmetic or therapeutic reasons.

The demineralized bone material according to the present disclosure may also be used to correct bone defects in orthopedic, neurosurgical plastic, or reconstructive surgery, in periodontal procedures, and in endodontic procedures. Examples include, but not limited to, repair of simple and compound fractures and non-unions, external and internal fixations, joint reconstructions such as arthrodesis, general arthroplasty, cup arthroplasty of the hip, femoral and humeral head replacement, femoral head surface replacement and total joint replacement, repairs of the vertebral column including spinal fusion and internal fixation, tumor surgery, e.g. deficit filling, discectomy, laminectomy, excision of spinal cord tumors, anterial cervical and thoracic operations, repair of spinal injuries, scoliosis, lordosis and kyphosis treatments, intermaxillary fixation of fractures, mentoplasty, temporomandibular joint replacement, alveolar ridge augmentation and reconstruction, inlay bone grafts, implant placement and revision, sinus lifts, etc. The standard surgical and dental procedures are suitable for use with the various methods as disclosed in Chen, et al., U.S. Pat. No. 6,180,606, and Dowd, et al., U.S. Pat. No. 5,507,813.

The following examples are illustrative of the methods for preparing a demineralized bone material with a flexible web-like section that is resistant to compression and extrusion according to the present disclosure, and orthopedic applications with the demineralized bone material so obtained.

### EXAMPLES

### Example 1

### Demineralization and Compression of Bone Material

Cortical bone is processed at an AATB-certified tissue bank where the bone is cleaned by removing adherent tissues. The bone is milled or cut into strips of various lengths, widths, and thicknesses as exemplified in FIG. 3A. The bone is demineralized in 0.5 M hydrochloric acid bath. The de-calcified bone is rinsed with deionized/distilled water. The bone is then placed in a sodium base until the runoff rinse solution reaches a neutral pH level. It is then rinsed again with deionized/disteilled water.

The demineralized bone (contains approximately <1% calcium) is then placed in a hydraulic press with one plastic plate and one metal plate and compressed to create a mechanically macerated flexible web-like section as shown in FIG. 3B.

### Example 2

### Freeze-Dry of Demineralized, Compressed Bone Material

The demineralized bone material is prepared from strips of cortical bone according to Example 1. After compression with a hydraulic press, the compressed bone material is placed inside of a sterilized Tyvek® Mylar® package. Moisture is withdrawn from the package by lyophilization and is removed until the moisture content in the demineralized bone is less than about 6% by weight.

### Example 3

### Preparation of Bone Graft Combination

A bone-repairing composition is prepared by mixing 20 grams of the demineralized bone prepared according to the method described in Example 1 and a carrier comprising 100 grams of saline. The bone graft composition is placed into a tubular shaped mold. The final dried composition is formed into a cylinder having a diameter of 1.25 cm and a base height of 2.5 cm. This is then rehydrated with a platelet concentrate to infiltrate the freeze-dried graft combination.

While the invention has been described in the specification and illustrated in the examples with reference to various embodiments, it will be understood by those skilled in the art that various changes may be made and equivalents may be substituted for elements thereof without departing from the scope of the invention as defined in the claims. Furthermore, the mixing and matching of features, elements and/or functions between various embodiments is expressly contemplated herein so that one of ordinary skill in the art would appreciate from this disclosure that features, elements and/or functions of one embodiment may be incorporated into another embodiment as appropriate, unless described otherwise above.

What is claimed is:

1. A method for preparing a demineralized bone material useful for orthopedic applications, comprising:
a) demineralizing a strip of bone;
b) compressing the strip of bone in a press to create a flexible web-like section; and
c) preserving the strip of bone.

2. The method of claim 1, wherein the strip of bone is obtained from an animal source or a human source.

3. The method of claim 1, wherein the strip of bone is obtained from cortical, cancellous and/or corticocancellous bone.

4. The method of claim 1, wherein the strip of bone is obtained lengthwise along a longitudinal axis of the bone.

5. The method of claim 1, wherein the strip of bone is preserved by freeze-drying.

6. The method of claim 1, wherein the strip of bone is preserved by freezing.

7. The method of claim 1, wherein the strip of bone is preserved by liquid storage.

8. The method of claim 1, wherein the strip of bone is compressed with a hydraulic press comprising two metal press plates or a plastic press plate opposed from a metal press plate to create the flexible web-like section.

9. The method of claim 8, wherein at least one of the two metal press plates or one of the plastic press plate and the metal press plate includes a textured surface to form a textured area on the demineralized bone material.

10. The method of claim 1, further comprising shaping and forming the demineralized bone material to fit a particular bone void.

11. A demineralized bone material with flexible web-like section that is resistant to compression and extrusion obtained by the method of claim 1.

12. A composition useful for orthopedic applications comprising the demineralized bone material of claim 11 and at least one carrier.

13. The composition of claim 12, wherein the at least one carrier is a phospholipid.

14. The composition of claim 12, wherein the at least one carrier is lecithin.

15. Bone implants or bone grafting materials comprising the demineralized bone material obtained by the method of claim 1 or a composition comprising said demineralized bone material.

16. An implant for use in bone reconstruction comprising the demineralized bone material obtained by the method of claim 1 or a composition comprising said demineralized bone material.

17. A method for preparing a bone void for orthopedic applications, comprising:
a) obtaining a demineralized bone material according to the method of claim 1 or a composition comprising said demineralized bone material;
b) shaping and/or forming the demineralized bone material into a bone void; and
c) optionally rehydrating the demineralized bone material.

18. The method of claim 17, wherein the demineralized bone material is rehydrated with an aqueous solution or fluid containing water.

19. The method of claim 17, wherein the demineralized bone material is rehydrated with corpus fluids, blood, or blood components.
